(19) United States
(12) Patent Application Publication
(10) Pub. No.: US 2023/0232868 A1
(43) Pub. Date: Jul. 27, 2023

(54) OAT-BASED PRODUCTS WITH HIGH OAT PROTEIN CONTENT AND FUNCTIONALITY AND PRODUCTION PROCESSES THEREOF

(71) Applicant: SOCIETE DES PRODUITS NESTLE S.A., Vevey (CH)

(72) Inventors: JULIANA VALLE COSTA SILVA, Fribourg (CH); NICOLE REBEKKA ROHRER, Reichenbach (CH); CHRISTOPH THOMAS WIDMER, Kehrsatz (CH); JAN BIEHL, Thun (CH); KLAUS INDERMUHLE, Thun (CH)

(21) Appl. No.: 18/003,703

(22) PCT Filed: Jul. 1, 2021
(86) PCT No.: PCT/EP2021/068148

§ 371 Date: Dec. 29, 2022

(30) Foreign Application Priority Data
Jul. 2, 2020 (EP) 20183828.1

(51) Int. Cl. A23L 2/66; A23L 2/74; A23L 2/84; A23L 7/10; A23L 29/30; C12N 9/24

(52) U.S. Cl. CPC A23L 2/66; A23L 2/74; A23L 2/84; A23L 7/115; A23L 29/30; C12N 9/2402; C12Y 302/01001

(57) ABSTRACT

The present disclosure provides a novel and gentle process for producing oat-based products containing oat proteins that have good functional properties, including solubility, emulsifying, foaming and gelling properties. This process includes hydrolysis to break down carbohydrates, physical separation to remove insoluble fibers, and membrane filtration to concentrate oat proteins by the removal of sugars. The method can include providing an oat mixture; hydrolyzing the oat mixture with an enzyme or a combination of enzymes; physically separating an insoluble material from the hydrolyzed oat mixture to form a soluble hydrolyzed oat mixture; applying membrane filtration to the soluble hydrolyzed oat mixture using a membrane having molecular weight cut-offs (MWCO) greater than 100 kDa.

### BACKGROUND

Due to concerns of, for example, allergies, lactose intolerance, calories, and cholesterol, many consumers need or prefer dairy alternatives/replacement food/drink products. In recent years, plant proteins have received a lot of attention as alternative protein sources in food/drink formulations due to their sustainability, low production cost, and health benefits. Protein food/drink products using plant sources, such as soy, pea, oat, rice, coconut, almond, cashew and hemp, are commercially available.

Oats are well received by consumers who have or prefer a healthy lifestyle and have been consumed in many different forms, such as rolled oats, bread, biscuits, cookies, cereals, etc. Oats have a very pleasant and round taste as well as much better sensorial attributes compared to many other plant protein sources.

However, limitations such as low availability and poor functionality have hindered wide applications of oat proteins. Commercial oat-based beverages usually contain very low amounts of oat proteins (from 0 to 1%), which makes them poor options as dairy alternatives. The low protein content in commercial oat-based beverages is mainly because these beverages are usually produced from oat flours, which have a low protein content (<13%). To increase the protein content, a high amount of oat flours can be used for production, but this would result in stability issues of the produced beverages.

Commercially available oat protein concentrates containing high oat protein contents can also be used to produce oat-based beverages. However, these commercial oat protein concentrates can have a very poor stability in water, and thus their applicability for liquid and semi-solid products are limited. This is mainly because the existing processes used to isolate the oat protein from the raw material impact the oat protein structure, thereby drastically reducing protein solubility and other functional properties (e.g., emulsifying, foaming and gelling properties) of the oat protein.

Thus, the final products using these commercial oat protein concentrates can have sandy mouthfeel and sedimentation, which are not preferred by consumers.

### SUMMARY

The present disclosure provides a novel and gentle process for producing oat-based products containing oat proteins that have good functional properties, including solubility, emulsifying, foaming and gelling properties. This process includes hydrolysis to break down carbohydrates, physical separation to remove insoluble fibers, and membrane filtration to concentrate oat proteins by the removal of sugars.

In an embodiment, a method for producing an oat-based product comprises providing an oat mixture, preferably having a total solids (TS) of 10-25%; hydrolyzing the oat mixture with an enzyme or a combination of enzymes, preferably at a temperature between 60° C. and 70° C., more preferably at 60° C.; physically separating an insoluble material from the hydrolyzed oat mixture by decantation and/or sieving to form a soluble hydrolyzed oat mixture; applying membrane filtration, preferably at a temperature from 50° C. to 60° C., to the soluble hydrolyzed oat mixture using a membrane having molecular weight cut-offs (MWCO) greater than 100 kDa, preferably greater than or equal to 500 kDa, to obtain a retentate.

In a particular preferred embodiment the membrane filtration is applied, preferably at a temperature from 50° C. to 60° C., to the soluble hydrolyzed oat mixture at TS from about 10% to about 15%, preferably about 10%. More preferably this membrane filtration is using a membrane having molecular weight cut-offs (MWCO) greater than 100 kDa, preferably greater than or equal to 500 kDa, to obtain a retentate.

In an embodiment, the hydrolyzed oat mixture is homogenized after the hydrolysis of the oat mixture, preferably at 60° C. and 200/50 bars.

In an embodiment, physically separated insoluble fibers are freeze-dried or spray dried.

In an embodiment, a permeate is obtained from the membrane filtration and steamed and/or evaporated,

In an embodiment to produce an oat-based ready-to-drink (RTD) product, at least one of sugar, preferably sucrose; the evaporated permeate; oil, preferably sunflower oil; buffer salts and sources of calcium, preferably dipotassium phosphate and tricalcium phosphate; or flavor enhancers, preferably sodium chloride is added to the retentate to form a mixture. Additionally, an ultra-high temperature (UHT) treatment including up or downstream homogenization can also be applied to the mixture; and the heat-treated mixture is then aseptically packaged.

In an embodiment, the oat mixture comprises an oat raw material selected from the group consisting of oat flour, defatted oat flour, oat flakes, and mixtures thereof; preferably defatted oat flour.

In an embodiment, the enzyme comprises at least one of amylase, amyloglucosidase, or β-glucanase; preferably amylase(s); or amylase and β-glucanase; or α-amylase and amyloglucosidase; or amylase, β-glucanase, and amyloglucosidase. In an embodiment, a total amount of the enzyme used for hydrolyzing the oat mixture is from 0.05 wt % to 0.3 wt %, preferably 0.2 wt %, of the oat raw material.

In an embodiment, the enzyme is inactivated at 90° C. to 95° C. for 10-15 minutes, and the hydrolyzed oat mixture is cooled to 20-60° C., preferably 60° C., after the inactivation of the enzyme.

In an embodiment, the membrane comprises a hydrophilic, hydrophobic, or an inorganic membrane, more preferably a ceramic membrane.

In an embodiment, an oat protein concentrate produced by the present process comprises 5-10 wt % of oat proteins; 2-7 wt % of fat; 5-20 wt % of carbohydrates; 2-4 wt % of sugars, mostly maltose; and 1-2 wt % of total dietary fibers comprising (i) 1-1.5 wt % of insoluble fibers and (ii) up to 0.5 wt % of soluble fibers comprising β-glucans, wherein the oat protein concentrate has a TS of 20-30%.

In an embodiment, the oat proteins in the oat protein concentrate has a solubility of 20% at pH 4 and of 83-86% from pH 7 to pH 11.

In an embodiment, the present disclosure provides an oat-based ready-to drink (RTD) beverage comprising the oat protein concentrate; and optionally at least one of sugar, preferably sucrose; the evaporated permeate; oil, preferably sunflower oil; buffer salts and sources of calcium, preferably dipotassium phosphate and tricalcium phosphate; or flavor enhancers, preferably sodium chloride.

In an embodiment, the oat RTD beverage comprises at least 3 wt %, preferably 3 to 5 wt % of oat proteins and/or 3-5 g oat proteins/100 ml of the oat RTD beverage; 3-5 g sugars/100 ml of the oat RTD beverage; and 1.5-3 g fat/100 ml of the oat RTD beverage. The oat RTD beverage can have a viscosity of 15-40 mPa·s (25° C., 100 s⁻¹).

In an embodiment, an oat syrup prepared from the permeate of the present process comprises up to 1 wt % of oat proteins; up to 0.1 wt % of fat; 40-50 wt % of carbohydrates; 20-30 wt % of sugars, mostly maltose; and up to 0.5 wt % of fibers. The oat syrup can have a TS of 65-80%.

In an embodiment, a natural fiber product prepared by the present process may have a composition of (i) 4-10 wt % of oat proteins; 1-4 wt % of fat; 5-20 wt % of carbohydrates; 2-3 wt % of sugars, mostly maltose; and 5-20 wt % of total dietary fibers comprising about 0.1-1 wt % of soluble fibers (beta-glucan), wherein the natural fiber product has a TS of 15-50%; and/or (ii) 15-30 wt % of oat proteins; 3-10 wt % of fat; 20-40 wt % of carbohydrates; 4-16 wt % of sugars; and 16-60 wt % of total dietary fibers comprising 15-56 wt % of insoluble fibers and 1-4 wt % of soluble fibers, wherein the natural fiber product has a TS of 90-98%.

An advantage of the present disclosure is to provide a method for producing oat-based products that contain highly functional oat proteins with high stability, solubility, gelation, emulsification, and foaming properties.

Another advantage of the present disclosure is to provide a method for producing oat-based products that have smooth taste and mouthfeel.

Yet another advantage of the present disclosure is to provide a method for producing oat-based products that have a high oat protein content.

An additional advantage of the present disclosure is to provide a method for producing oat-based products that have a controlled sugar content; i.e., sugar generation by modulation of the enzymatic hydrolysis step and/or sugar removal by modulation of the membrane filtration step.

Another advantage of the present disclosure is to provide a method for producing oat-based products that have a modulated protein/sugar ratio.

Still another advantage of the present disclosure is to provide dairy alternative protein products that contain highly functional proteins with high stability, solubility, gelation, emulsification, and foaming.

Another advantage of the present disclosure is to provide dairy alternative protein products that have smooth taste and mouthfeel.

Yet another advantage of the present disclosure is to provide dairy alternative protein products that have a high protein content.

An additional advantage of the present disclosure is to provide dairy alternative protein products that have a modulated protein/sugar ratio.

Additional features and advantages are described herein, and will be apparent from, the following Detailed Description and the figures.

### BRIEF DESCRIPTION OF THE FIGURES

FIG. 1 shows the protein solubility profile of the oat protein concentrate produced by the present process as a function of pH compared to a commercial oat protein concentrate.

FIG. 2 shows the foaming volume profile of the oat protein concentrate produced by the present process as a function of time compared to a commercial oat protein concentrate.

FIG. 3 shows the RVA profiles of oat flour in water (TS=15%) during hydrolysis with different enzymes.

FIG. 4 shows a zoom-in of the RVA profiles of oat flour in water (TS=15%) during hydrolysis with different enzymes shown in FIG. 3.

FIG. 5 shows the flux during flat sheet membrane filtration of samples produced with different TS values where BAN™480 L was used either alone or in combination with Termamyl®. All samples were treated with 0.2 wt % enzyme(s) per weight of oat flour. BAN480L and Termamyl® were combined in a 1:1 ratio.

FIG. 6 shows the permeate flux during flat sheet membrane filtration (MWCO=500 kDa) of different hydrolyzed oat mixtures made using a decanter at different initial TS values.

### DETAILED DESCRIPTION

The various aspects and embodiments according to the present disclosure, as set forth herein, are illustrative of the specific ways to make and use the invention and do not limit the scope of invention when taken into consideration with the claims and the detailed description. It will also be appreciated that features from aspects and embodiments of the invention may be combined with further features from the same or different aspects and embodiments of the invention.

As used in this detailed description and the appended claims, the singular forms “a,” “an” and “the” include plural referents unless the context clearly dictates otherwise. For example, reference to “an ingredient” or “a method” includes a plurality of such “ingredients” or “methods.” The term “and/or” used in the context of “X and/or Y” should be interpreted as “X,” or “Y,” or “X and Y.” Similarly, “at least one of X or Y” should be interpreted as “X,” or “Y,” or “both X and Y.” Similarly, the words “comprise,” “comprises,” and “comprising” are to be interpreted inclusively rather than exclusively. Likewise, the terms “include,” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of” and “consisting of” the disclosed components. “Consisting essentially of” means that the embodiment or component thereof comprises more than 50 wt. % of the individually identified components, preferably at least 75 wt. % of the individually identified components, more preferably at least 85 wt. % of the individually identified components, most preferably at least 95 wt. % of the individually identified components, for example at least 99 wt. % of the individually identified components.

All ranges described are intended to include all numbers, whole or fractions, contained within the said range. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth. As used herein, wt. % refers to the weight of a particular component relative to total weight of the referenced composition. Material that passes through a membrane is called “permeate”; material that does not pass through a membrane and is recirculated is called “retentate”.

In the context of the invention, the term “oat-based products” refers to the products which comprise oat proteins and which are obtained by the process of the invention. Depending on the final step, it refers to the retentate (final step=membrane filtration) or to ingredients/food products, e.g. oat-based RTD beverage, derived from the further processing of the retentate obtained after membrane filtration. Additionally, it may also refer to the products which comprise oat fibers and/or carbohydrates and which are obtained by the process of the invention. In particular, it may refer to the permeate which is obtained after the membrane filtration step and which may be optionally steamed and/or evaporated after the membrane filtration step. Alternately, it may refer to the physically separated insoluble fibers which are obtained after decantation and/or sieving and which may be optionally freeze dried or spray dried after decantation and/or sieving. In a preferred embodiment, the term “oat-based products” refers to the retentate or to the oat-based RTD beverage. More preferably, it refers to the retentate.

All percentages expressed herein are by weight of the total weight of the composition unless expressed otherwise. When reference is made to the pH, values correspond to pH measured at 25° C. with standard equipment.

“Astringency” is generally recognized as a feeling of puckering and dryness in the palate and is known to build in intensity and become increasingly difficult to clear from the mouth over repeated exposures. Astringency is a dry sensation experienced in the mouth and is commonly explained as arising from the loss of lubricity owing to the precipitation of proteins from the salivary film that coats and lubricates the oral cavity. Astringency is not confined to a particular region of the mouth but is a diffuse surface phenomenon, characterized by a loss of lubrication, which takes a time of the order of 15-20 seconds to develop fully. Therefore, astringency is quite different from the more well-known taste sensations.

A “ready to drink” beverage or “RTD” beverage is a beverage in liquid form that can be consumed without further addition of liquid. Preferably an RTD beverage is aseptic.

A “liquid concentrate” or “concentrate” is a liquid that is formulated to be diluted before administration. Further in this regard, the liquid concentrates disclosed herein are only administered after addition of another ingredient, such as a liquid diluent.

The present disclosure provides a novel and gentle process for producing oat-based products containing proteins that have good solubility as well as other functional properties. This process includes hydrolysis to break down carbohydrates, physical separation to remove insoluble fibers, and membrane filtration to concentrate oat proteins by the removal of sugars.

In a general embodiment, the present disclosure provides a process of producing an oat-based product. The process comprises hydrolyzing an oat mixture with an enzyme, physically separating an insoluble material from the hydrolyzed oat mixture by decantation and/or sieving to form a soluble hydrolyzed oat mixture, and applying membrane filtration to the soluble hydrolyzed oat mixture using a membrane having molecular weight cut-offs (MWCO) greater than 100 kDa, preferably greater than or equal to 500 kDa, to obtain a retentate.

The oat mixture may comprise an oat raw material, such as oat flour, defatted oat flour, oat flakes, or mixtures thereof. In one embodiment, the oat flour used may contain about 10-15 wt % proteins, about 4-6 wt % fat, about 55-70 wt % carbohydrates, and about 8-11 wt % fibers. The oat flour can have a TS of about 90-95 wt %. The oat mixture preferably comprises defatted oat flour, which can lead to a high oat protein recovery yield over the process. For example, the defatted oat flour may contain less than about 4 wt % fat, preferably less than about 3 wt % fat.

The oat raw material may be initially mixed with water to prepare the oat mixture. The oat mixture may have a total solids (TS) of 10≤TS≤25% before the subsequent membrane filtration, preferably 10≤TS≤15%. For example, the oat mixture can have a TS of about 10%, 15%, 20%, or 25%. The mixture can be diluted to have a desirable TS before the membrane filtration. The oat mixture preferably has a TS from about 10% to about 15% before the membrane filtration, for example, about 10% or about 15%, preferably about 10%.

The oat mixture can then be hydrolyzed by an enzyme or an enzyme mixture at a suitable temperature, for example, between about 60° C. and about 70° C., preferably at about 60° C. At the suitable temperature, the starch in the oat mixture starts the process of gelatinization. The temperature of this step is chosen based on the optimal temperatures for the activity of the enzyme(s) being used.

The enzymatic hydrolysis step can be performed for a suitable period of time depending on factors such as the enzyme(s) used, enzyme concentration, and/or the conditions of the hydrolysis (time, temperature). In some embodiments, the enzymatic hydrolysis step can be performed for about 30 minutes or 2 hours, preferably for 1 hour. In another preferred embodiment, the enzymatic hydrolysis step is performed for 30 minutes to 2 hours, more preferably for 45 minutes to 90 minutes, most preferably for 45 minutes to 75 minutes.

The enzyme can comprise at least one of amylase, amyloglucosidase, or β-glucanase. The enzyme may comprise amylase; a mixture of amylase and β-glucanase; a mixture of α-amylase and amyloglucosidase; or a mixture of amylase, β-glucanase, and amyloglucosidase. Combinations of enzymes can lead to a higher oat protein recovery yields over the process compared to when amylase is used alone. Enzymatic hydrolysis with β-glucanase activity is needed to reduce viscosity and to avoid blocking the filtration membrane. This activity may be found through a specific enzyme or through a side activity of amylases. Hence, in a preferred embodiment, the enzyme comprises β-glucanase and/or amylase with β-glucanase side activity. When the enzyme does not comprise β-glucanase, it comprises amylase with β-glucanase side activity. In particular, the enzyme may comprise amylase with β-glucanase side activity; a mixture of amylase and β-glucanase; a mixture of α-amylase with β-glucanase side activity and amyloglucosidase; or a mixture of amylase, β-glucanase, and amyloglucosidase. It may also comprise a mixture of amylase with β-glucanase side activity and β-glucanase or a mixture of amylase with β-glucanase side activity, β-glucanase, and amyloglucosidase.

In a preferred embodiment, the enzyme is free from protease or enzymes with protease side activity. Indeed, it is advantageous that the oat proteins remain intact and are not broken down into peptides. In particular, the oat proteins should remain intact to be collected and concentrated in the retentate. If they are broken down into peptides, peptides would pass through the membrane during the membrane filtration step and would be collected in the permeate which also comprises undesirable compounds (e.g. carbohydrates). Moreover, without wishing to be bound by theory, the hydrolysis of oat proteins by protease would generate an undesirable bitterness which would be unpleasant, in particular for food applications. A total amount of the enzyme used for hydrolyzing the oat mixture can be from about 0.05 wt % to about 0.3 wt %, for example, from about 0.05 wt % to about 0.2 wt %, from about 0.1 wt % to about 0.3 wt %, from about 0.1 wt % to about 0.2 wt %, from about 0.15 wt % to about 0.25 wt %,

preferably about 0.2 wt %, of the oat raw material. Preferably, a total amount of about 0.2 wt % enzyme of the oat raw material is used for the hydrolysis. Lower dosages of enzymes may lead to lower oat protein recovery yields.

The enzyme(s) are added to hydrolyze the carbohydrate fraction, such as starch and/or fiber of the oat raw material. In particular, the enzyme(s) hydrolyze soluble fibers mainly into mono- or disaccharides (e.g. maltose). This step reduces the viscosity of the oat mixture to facilitate its processability and to avoid fouling during the membrane filtration step.

Optionally, a homogenization step can be applied to break down large particles present in the oat mixture after the hydrolysis step. The homogenization step can be applied at 60° C. This step can lead to an increase in the oat protein recovery yield compared to a process where this step was not performed.

The homogenization step can be applied at a temperature, for example, from about 40° C. to about 80° C., preferably from about 50° C. to about 70° C., more preferably from about 55° C. to about 65° C., and most preferably at about 60° C. The homogenization step is preferably applied at 200/50 bars.

An enzyme inactivation step can then be applied in order to stop the enzyme(s) activity. Otherwise, the enzyme(s) may remain active in the final product, which may lead to food quality issues. The enzyme inactivation step can be applied at a temperature from about 90 to about 95° C. This step can be applied for a time period of about 10-15 minutes.

The hydrolyzed oat mixture can be cooled to about 20-60° C. after the inactivation of the enzyme. Preferably, the hydrolyzed oat mixture is cooled to a temperature of 60° C.

High amounts of insoluble fiber can lead to extensive fouling during the membrane filtration step. Thus, a physical separation step can then be applied to remove the insoluble material from the hydrolyzed oat mixture. Centrifugation, decantation, and/or sieving can be used for this step to obtain a soluble hydrolyzed oat mixture. When centrifugation is used, a pH adjustment step is required before the physical separation step to avoid protein precipitation. This pH adjustment step is not required when decantation or sieving is used.

Preferably, decantation is used for this step. Decantation can lead to efficient removal of insoluble materials as well as a high oat protein recovery yield over the process. In some embodiments, the protein recovery rate from decantation can be about 80-90%.

In a preferred embodiment, the process of the invention does not comprise any additional decantation step after the physical separation step. Especially, when the physical separation step is performed by decantation, the process of the invention comprises a single decantation step. A decanter centrifuge is based on the principle of sedimentation in a liquid medium which is accelerated by a centrifugal force. The decanter distinguishes itself from other centrifuges by a continuous removal of the sediment by an axial screw conveyor. The difference in speed between the bowl and the conveyor is called the differential speed, which makes the solids move to the solids discharge. The differential speed impacts the residence time of the product and the dryness of the final cake that leaves the machine at the solids discharge. When compared to other liquid-solid separation techniques, the decanter centrifuge can often run at higher volumes in a continuous design. Moreover, the solids that leave the decanter centrifuge often have a lower water content due to the screw that presses the last bit of water out of the solids.

The pH of the oat hydrolysate, without adjustment, can be below 7, for example, about 6.3. If centrifugation is used, the pH value of the hydrolyzed oat mixture is first adjusted to an alkaline pH, such as a pH of about 8, before centrifugation to increase protein solubility and subsequently to prevent protein precipitation during the centrifugation step. The final pH can be between 7 and 10, preferably between 8 and 10. Lower pH values can also lead to a significant decrease in the oat protein recovery yield over the process when centrifugation is used, due to the low solubility of oat proteins at lower pH values. If decantation or sieving is used for this physical separation step, no pH adjustment is necessary. In particular, if decantation or sieving is used for this physical separation step, the retentate has a pH of 6.0 to 7.0, preferably of 6.2 to 6.6. If the oat raw material comprises oat flakes, the insoluble material can be physically separated from the hydrolyzed oat mixture by sieving.

Insoluble fibers can be separated and obtained from the physical separation step and can be used as and/or in a natural fiber product, which can be used in food product. The obtained insoluble fibers can comprise about 4-10 wt % of oat proteins; about 1-4 wt % of fat; about 5-20 wt % of carbohydrates; about 2-3 wt % of sugars, mostly maltose; and about 5-20 wt % of total dietary fibers comprising about 0.1-1 wt % of soluble fibers (beta-glucan). The obtained insoluble fibers can have a TS of about 15-50%.

In one embodiment, the obtained insoluble fibers comprise about 8 wt % of oat proteins; about 2 wt % of fat; about 10 wt % of carbohydrates; about 2-3 wt % of sugars, mostly maltose; and about 10 wt % of total dietary fibers comprising about 0.5 wt % of soluble fibers (beta-glucan). The obtained insoluble fibers can have a TS of about 30%.

The insoluble fibers can be freeze-dried or spray-dried to produce a dry fiber product. The dried insoluble fibers can comprise about 15-30 wt % of oat proteins; about 3-10 wt % of fat; about 20-40 wt % of carbohydrates; about 4-16 wt % of sugars; and about 16-60 wt % of total dietary fibers comprising about 15-56 wt % of insoluble fibers and about 1-4 wt % of soluble fibers. The dried fiber product can have a TS of about 90-98%.

In one embodiment, the dried insoluble fibers comprise about 25 wt % of oat proteins; about 7 wt % of fat; about 30 wt % of carbohydrates; about 8 wt % of sugars; and about 32 wt % of total dietary fibers comprising about 30 wt % of insoluble fibers and about 2 wt % of soluble fibers. The dried fiber product can have a TS of about 95%.

The soluble hydrolyzed oat mixture obtained from the physical separation step may have a TS of about 10-25%. For example, the soluble hydrolyzed oat mixture can have a TS of about 10%, 15%, 20%, or 25%. The soluble hydrolyzed oat mixture preferably has a TS from about 10% to about 15%, for example, about 10% or about 15%. If the TS of the soluble hydrolyzed oat mixture is above about 15%, the soluble hydrolyzed oat mixture can be diluted to have a more preferable TS for the subsequent membrane filtration step.

The membrane filtration step can then be applied to the soluble hydrolyzed oat mixture to obtain a retentate and a permeate. Membrane filtration is a pressure driven process in which the feed solution, the solution to be concentrated or fractionated, is forced through the membrane. Two fractions are obtained by this process: a retentate, which corresponds to the retained liquid; and a permeate, which corresponds to the liquid passing through the membrane.

This membrane filtration step allows the concentration of large molecules, e.g., proteins and remaining large carbohydrates, which are retained in the retentate, and the removal of small molecules, e.g., hydrolyzed carbohydrates, that pass through the membrane and are then collected in the permeate.

The membranes used in this step preferably have large molecular weight cut-offs (MWCO)/pore sizes to ensure a high protein content in the retentate and permeate flux.

The use of lower MWCOs can led to excessive fouling during the membrane filtration process and subsequently to a significant decrease in the oat protein content in the final retentate. The MWCOs of the membranes used can be 100 kDa or greater, for example, between about 100 kDa and about 0.2 μm, preferably greater than or equal to 500 kDa.

Flat sheets or spiral wound membranes can be used in this step. Different types of membrane materials can be used. For protein concentration applications, a hydrophilic and non-protein binding material is usually preferable. The membrane material used in the present process can be a hydrophilic, hydrophobic, and/or an inorganic material. In one embodiment, the membrane material is preferably ceramic. An adapted membrane material, such as ceramic, can significantly increase the protein content in the retentate and permeate flux.

The membrane filtration step can be carried out at a temperature greater than 50° C., preferably from about 50° C. to about 60° C. Temperatures lower than 10° C. may lead to excessive fouling due to high viscosity of the hydrolyzed oat mixture during the membrane filtration step. Temperatures between 10 and 50° C. may cause microbiological growth. Using higher temperatures, p.e. 60° C., can significantly increase the protein content in the retentate and permeate flux and are preferred in case the membrane material is not thermo-sensitive.

Different types of membrane filtration, such as microfiltration and ultrafiltration, can be used. Each membrane type has pores with different sizes.

In some embodiments, ultrafiltration can be used. “Ultrafiltration” is a membrane filtration technique using hydrostatic pressure to force a liquid through a semi-permeable membrane. Suspended solids and high molecular weight solutes are retained in ultrafiltration, while water and low molecular weight solutes cross the membrane. Ultrafiltration is used in industry and research to purify and concentrate solutions containing large molecular weight molecules (10³-10⁶ Da). Ultrafiltration allows an efficient and, at the same time, gentle separation of large molecular weight compounds. Any common type of ultrafiltration membrane may be used in the ultrafiltration, and suitable ultrafilters are commercially available, for example from Millipore Corp. and Desal Systems. Techniques by which ultrafiltration may be performed include flat, spiral, and hollow fiber techniques, for example. The ultrafiltration may be performed in various modes, such as dead-end, crossflow and back-flush operating modes. The present disclosure is not limited to a specific embodiment of the ultrafiltration membrane, the ultrafiltration technique or the ultrafiltration mode.

In some embodiments, microfiltration can be used. “Microfiltration” is filtration that uses a membrane having a pore size range from 0.1 to 10 μm and for which pressurization is optional. The microfiltration can use membranes that are hollow fibers, a flat sheet, tubular, spiral wound, hollow fine fibers or track etched, for example. The present disclosure is not limited to a specific embodiment of the microfilter.

In a preferred embodiment, the process of the invention does not comprise any step of sterilization, i.e. a step of heat treatment at temperatures above 100° C., before the membrane filtration step. Indeed, sterilization would lead to the aggregation/denaturation of the oat proteins which would negatively affect the functionality (e.g. solubility) of oat proteins, especially for RTD beverage applications.

A retentate can be obtained after the membrane filtration step. The retentate contains highly functional oat proteins and can be used in and/or as an oat protein concentrate. In an embodiment, the oat protein concentrate can comprise about 5-10 wt % of oat proteins; about 2-7 wt % of fat; about 5-20 wt %, for example, about 10 wt % of carbohydrates; about 2-4 wt % of sugars, mostly maltose; and about 1-2 wt % of total dietary fibers comprising (i) about 1-1.5 wt % of insoluble fibers and (ii) up to about 0.5 wt % of soluble fibers comprising β-glucans. The oat protein concentrate can have a TS of about 20-40%, for example, about 25-40% or about 20-30%. In a preferred embodiment, in the context of the invention, the oat protein concentrate is a retentate obtained by the process according to the invention. In a more preferred embodiment, the retentate can comprise about 5-10 wt % of oat proteins; about 2-7 wt % of fat; about 5-20 wt %, for example, about 10 wt % of carbohydrates; about 2-4 wt % of sugars, mostly maltose; and about 1-2 wt % of total dietary fibers comprising (i) about 1-1.5 wt % of insoluble fibers and (ii) up to about 0.5 wt % of soluble fibers comprising β-glucans. The retentate can have a TS of about 20-40%, for example, about 25-40% or about 20-30%.

Preferably, the oat proteins of the retentate or the oat protein concentrate are intact. Oat proteins which are intact have good functionality, such as improved solubility compared to aggregated/denatured oat proteins.

In a preferred embodiment, the retentate has a pH of 6.0 to 7.0, more preferably of 6.3 to 6.6. These pH ranges of the retentate contribute to preserve the functionality of oat proteins. In particular, if the pH of the retentate is below these ranges, it may lead to the aggregation of the proteins which may negatively impact the functionality (e.g. solubility) of oat proteins.

The oat protein concentrate contains highly functional oat proteins with high stability, solubility, gelation, emulsification, and foaming properties. The oat proteins in the oat protein concentrate can maintain their best performance due to the mild processing by membrane filtration. The oat protein concentrate has a high oat protein content, which is concentrated by membrane filtration. The oat protein concentrate also has a controlled sugar generation and/or sugar reduction due to the hydrolysis of carbohydrates and partial removal of the hydrolyzed carbohydrates (sugars) by membrane filtration, respectively. Further, the protein/sugar ratio of the oat protein concentrate can be modulated by process control and/or combination of different streams during/by the present process.

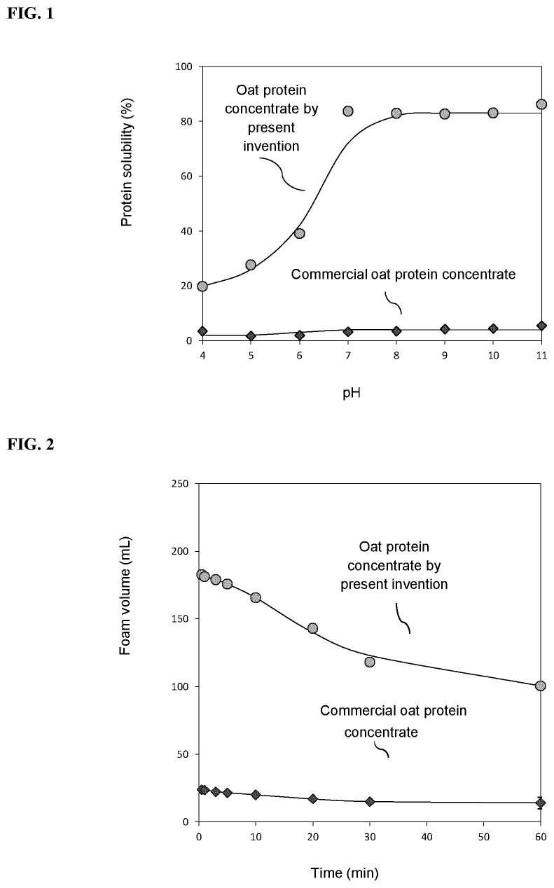

As shown in FIG. 1, compared to a commercial oat protein concentrate, the protein in the oat protein concentrate obtained by the present process has a greater solubility of about 20% or greater at a pH>=4. For example, at a pH >=5, the oat protein concentrate obtained by the present process has a protein solubility of about 30% or greater; and at a pH >=6, the oat protein concentrate obtained by the present process has a protein solubility of about 40% or greater. Notably, at a pH >=7, the oat protein concentrate obtained from the present process has a protein solubility of about 80%, which is much higher than that of the commercial oat protein concentrate, which is only up to about 5% at a pH of 4-11.

As shown in FIG. 2, compared to the commercial oat protein concentrate, the protein in the oat protein concentrate obtained by the present process has greater foaming properties. During the whole time measured, the oat protein concentrate obtained from the present process has a foaming volume of at least 4 times (about 100 mL or greater) of that of the commercial oat protein concentrate (up to 25 mL).

Additional ingredients can be added to the retentate or the oat protein concentrate to produce oat-based ready-to drink (RTD) beverages. For example, at least one of sugar, preferably sucrose; oil, preferably sunflower oil; buffer salts and sources of calcium, preferably dipotassium phosphate and tricalcium phosphate; or flavor enhancers, preferably sodium chloride, can be added to the retentate. Sodium chloride is a flavor enhancer which contributes to the reduction of off-notes in the oat-based products (e.g. retentate, oat protein concentrate, oat-based RTD beverage), including bitterness and astringency. The buffer salts may also be disodium phosphate. The sources of calcium may also be calcium carbonate.

One or more vitamins and/or minerals can also be added to the retentate to produce the oat-based RTD beverages. Examples of minerals, vitamins and other micronutrients optionally present in the oat-based RTD beverages include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chloride, potassium, sodium, selenium, chromium, molybdenum, taurine, and L-carnitine.

Optionally, a permeate, rich in carbohydrates and/or hydrolytes thereof, can be obtained from the membrane filtration step. The permeate can be steamed and/or evaporated to obtain an evaporated permeate, which can be used in and/or as a side product of an oat syrup. The oat syrup can also be added to the retentate or the oat protein concentrate as an alternative source of sugar.

In one embodiment, the oat syrup can comprise up to about 1 wt % of oat proteins; up to about 0.1 wt % of fat; about 40-50 wt % of carbohydrates; about 20-30 wt % of sugars, mostly maltose; and up to about 0.5 wt % of fibers. The oat syrup can have a TS of about 65-80%.

A heat treatment, for example, an ultra-high temperature (UHT) treatment, including up or downstream homogenization, can be applied to the mixture of the retentate or the oat protein concentrate and the additional ingredient(s). The heat-treated mixture can then be aseptic packaged. The oat-based RTD beverage can be used in and/or as a dairy alternative product.

In one embodiment, the oat-based RTD beverage can comprise at least about 3 wt %, preferably about 3 to 5 wt % of oat proteins and/or 3-5 g oat proteins/100 ml of the oat RTD; about 3-5 g sugars/100 ml of the oat RTD; and about 1.5-3 g fat/100 ml of the oat RTD. The oat RTD can have a viscosity of 15-40 mPa·s (25° C., 100 s⁻¹). Preferably, the oat proteins of the oat-based RTD beverage are intact. Oat proteins which are intact have good functionality, especially for RTD beverage applications, such as improved solubility compared to aggregated/denatured oat proteins. The oat-based RTD beverage contains highly functional oat proteins with high stability, solubility, gelation, emulsification, and foaming properties. The oat proteins in the oat-based RTD beverage can maintain their best performance due to the mild processing by membrane filtration. The oat RTD has great sensory and nutritional performance. The oat-based RTD beverage has smooth taste and mouthfeel due to the high viscosity promoted by intact oat proteins and/or the right enzyme choice and/or salt addition. The oat-based RTD beverage has a high oat protein content, which is concentrated by membrane filtration. The oat-based RTD beverage also has a controlled sugar generation and/or sugar reduction due to the hydrolysis of carbohydrates and partial removal of the hydrolyzed carbohydrates (sugars) by membrane filtration, respectively. Further, the protein/sugar ratio of the oat-based RTD beverage can be modulated by process control and/or combination of different streams during/by the process.

In a preferred embodiment, the oat-based RTD beverage has a pH of 7 to 7.7, preferably a pH of 7.3 to 7.7, more preferably of 7.5. Especially, the pH of the beverage may be regulated by using a buffer salt, such as dipotassium phosphate or disodium phosphate. At this range of pH, the oat-based RTD beverage does not exhibit physical instability phenomenon over the shelf-life which may negatively impact the visual aspect and the organoleptic profile of the oat-based RTD beverage. For example, it does not exhibit protein aggregation and viscosity increase. In addition, at this range of pH, the off-notes, including astringency, of oat-based RTD beverage are reduced.

### EXAMPLES

### Example 1: Process

In an example process, an enzyme or an enzyme blend (0.2% weight of oat flour) was mixed with water, and oat flour was gradually added to reach a TS of 15%. The mixture was heated and kept at 60° C. for 1 h. The mixture was homogenized (200/50 bars, and preferably at 60° C.), heated to 90° C., hold at 90° C. for 10 min to inactivate the enzymes and cooled to 60° C. Then, the oat slurry was passed through a decanter (6000 rpm drum, 10 rpm screw, 500 L/h). The obtained heavy phase was oven dried at 105° C. for about 2-3 h and subsequently dry milled and sieved (mesh 1 mm). Membrane filtration was applied to the obtained light phase (i) with a spiral membrane made from regenerated cellulose (RC) with a 500 kDa MWCO and a membrane area of 11.4 m², at 50° C., with a flow of 3000-3800 L/h and a transmembrane pressure of about 1.2 bar; and (ii) alternatively, with a ceramic membrane with a 0.14 μm MWCO and a membrane area of 7.6 m², at 60° C., with a flow of 1900-3300 L/h and a transmembrane pressure of about 1.8 bar. The permeate obtained from the membrane filtration step was concentrated in a two-stage evaporator. The obtained retentate was mixed with other ingredients (e.g., water, sugar, sunflower oil, tricalcium phosphate, dipotassium phosphate) to produce the RTD product, followed by indirect UHT treatment and aseptic filling in bottles.

### Example 2: Enzymatic Hydrolysis

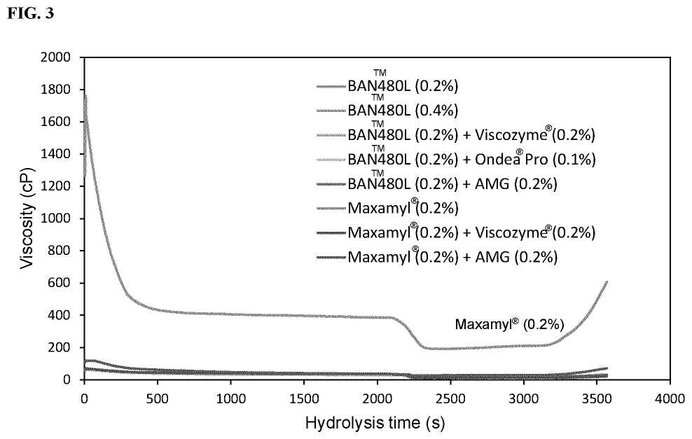

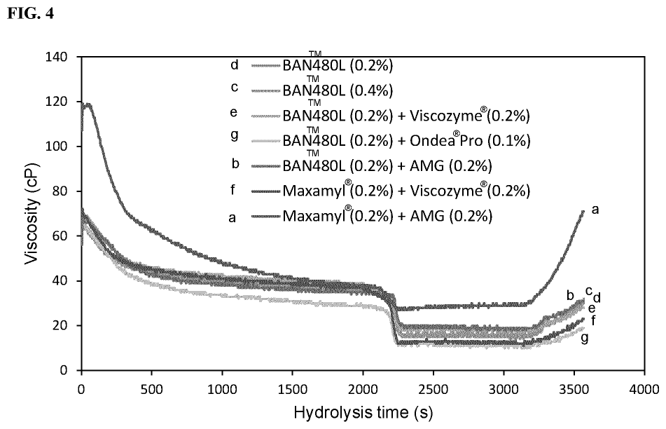

A Rapid Visco Analyzer (RVA) was used to simulate the hydrolysis and inactivation steps. Commercially available enzymes are used for the tests. FIG. 3 and FIG. 4 are the RVA profiles for the different enzymes tested. FIG. 3 shows that the α-amylase Maxamyl® without any other enzyme results in an oat slurry with the highest viscosity. FIG. 4 shows that Maxamyl® in combination with the amyloglucosidase (AMG) resulted in the highest viscosity compared to all the other samples in FIG. 4. The difference between the 2 α-amylases tested, BAN™ 480L and Maxamyl®, is the β-glucanase side-activity of BAN™ 480L. The cleavage of β-glucans has a big impact on the viscosity. This is also shown by the combination of Maxamyl® and the β-glucanase Viscozyme®, which has a similar viscosity profile as BAN™ 480L.

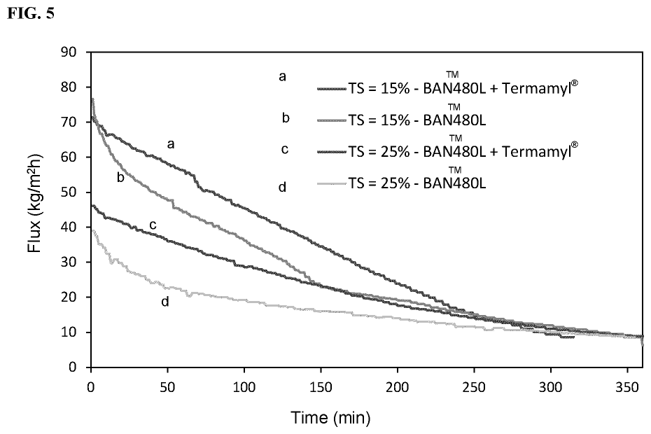

The combination of two α-amylases, BAN™ 480L and Termamyl®, was also investigated. Table 1 and FIG. 5 show the results.

TABLE 1
Results from trials carried out with different TS values where BAN ™
480L was used either alone or in combination with Termamyl ®.

| | | | | Protein | |
|---|---|---|---|---|---|
| | | | | on TS in | |
| | | TS (%) | TS (%) | the | TS of |
| | Initial | after | of the | retentate | permeate |
| | TS (%) | decantation | retentate | (%) | (%) |
| BAN ™ | 15 | 14.5 | 27.9 | 24.3 | 11.0 |
| 480L | | | | | |
| BAN ™ | 15 | 14.3 | 32.1 | 33.2 | 11.8 |
| 480L + | | | | | |
| Termamyl ® | | | | | |
| BAN ™ | 25 | 24.3 | 33.4 | 23.8 | 18.3 |
| 480L | | | | | |
| BAN ™ | 25 | 24.0 | 36.4 | 28.5 | 18.8 |
| 480L + | | | | | |
| Termamyl ® | | | | | |

These results indicate that the combination of BAN™ 480L with Termamyl® results in higher permeate flux (FIG. 5) and subsequently a higher protein content on TS in the retentate (Table 1).

### Example 3: Initial Total Solids (TS)

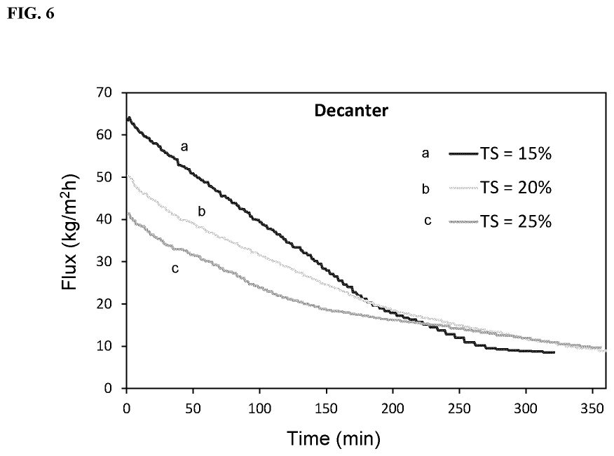

The initial TS of the oat slurry corresponds to the ratio of oat flour to water at the beginning of the process. Table 2 summarizes the results of trials with different initial TS values, and FIG. 6 shows the permeate flux of these trials during the membrane filtration step.

TABLE 2
Results from trials carried out with a decanter
and different initial TS values.

| | After | After membrane filtration | | |
|---|---|---|---|---|
| | mechanical | | Protein on | |
| | separation step | | TS in the | |
| | (decantation) | | retentate | TS of |
| Initial TS (%) | TS (%) | TS (%) | (%) | permeate (%) |
| 15 | 13.7 | 28.7 | 28.0 | 9.5 |
| 20 | 17.8 | 31.0 | 25.5 | 13.1 |
| 25 | 22.5 | 31.0 | 22.8 | 16.5 |

During membrane filtration, the permeate flux of samples with a higher TS was lower as shown in FIG. 6, as viscosity, concentration polarization and fouling are higher at a higher TS.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

### Example 4: RTD Beverage

The retentate obtained via membrane filtration in Example 1 was used to produce an oat-based RTD beverage containing 3% oat protein. The retentate was mixed with other ingredients (described in Table 3 below) for 30 minutes at ambient temperature. The mixture was then UHT treated at 145° C. for 6 seconds, homogenized at 200/50 bars at 78° C., cooled down to 20° C. and aseptic filled in plastic bottles. The oat-based RTD beverage has a pH of 7.5.

TABLE 3

| Ingredients | % in recipe |
|---|---|
| Retentate (from Example 1) | 60.0 |
| Brown Sugar | 3.00 |
| Tricalcium Phosphate | 0.35 |
| Dipotassium Phosphate | 0.80 |
| Sodium Chloride | 0.06 |

1. A process of producing an oat-based product, the process comprising:
providing an oat mixture;
hydrolyzing the oat mixture with an enzyme or a combination of enzymes;
physically separating an insoluble material from the hydrolyzed oat mixture by decantation and/or sieving to form a soluble hydrolyzed oat mixture; and
applying membrane filtration, preferably at a temperature from 50° C. to 60° C., to the soluble hydrolyzed oat mixture using a membrane having molecular weight cut-offs (MWCO) greater than 100 kDa.

2. The process of claim 1, wherein the oat mixture comprises an oat raw material selected from the group consisting of oat flour, defatted oat flour, oat flakes, and mixtures thereof.

3. The process of claim 1, wherein the enzyme(s) comprise amylase.

4. The process of claim 1, wherein a total amount of the enzyme used for hydrolyzing the oat mixture is from 0.05 wt % to 0.3 wt % of the oat raw material.

5. The process of claim 1 further comprising inactivating the enzyme at 90° C. to 95° C. for 10-15 minutes and cooling the hydrolyzed oat mixture to 20-60° C.

6. The process of claim 1, wherein the membrane comprises a ceramic membrane.

7. An oat protein concentrate comprising:
5-10 wt % of oat proteins;
2-7 wt % of fat;
5-20 wt % of carbohydrates;
2-4 wt % of sugars, mostly maltose; and
1-2 wt % of total dietary fibers comprising (i) 1-1.5 wt % of insoluble fibers and (ii) up to 0.5 wt % of soluble fibers comprising β-glucans, and
wherein the oat protein concentrate has a TS of 20-30%.

8. The oat protein concentrate of claim 6, wherein the oat proteins has a solubility of 20% at pH 4 and of 83-86% from pH 7 to pH 11.

9. An oat-based ready-to drink (RTD) beverage comprising
providing an oat mixture;
hydrolyzing the oat mixture with an enzyme or a combination of enzymes;
physically separating an insoluble material from the hydrolyzed oat mixture by decantation and/or sieving to form a soluble hydrolyzed oat mixture; and
applying membrane filtration, preferably at a temperature from 50° C. to 60° C., to the soluble hydrolyzed oat mixture using a membrane having molecular weight cut-offs (MWCO) greater than 100 kDa concentrate; and at least one of sugar.

10. The oat RTD beverage of claim 9 comprising:
at least 3 wt %, preferably 3 to 5 wt % of oat proteins and/or 3-5 g oat proteins/100 ml of the oat RTD beverage;
3-5 g sugars/100 ml of the oat RTD beverage; and
1.5-3 g fat/100 ml of the oat RTD beverage,
wherein the oat RTD beverage has a viscosity of 15-40 mPa·s (100 s⁻¹).

11-12. (canceled)
